(19) United States
(12) Patent Application Publication
(10) Pub. No.: US 2008/0097577 A1
(43) Pub. Date: Apr. 24, 2008

(54) MEDICAL DEVICE HYDROGEN SURFACE TREATMENT BY ELECTROCHEMICAL REDUCTION

(75) Inventors: Liliana Atanasoska, Edina, MN (US); Jan Weber, Maastrich (NL); Robert W. Warner, Woodbury, MN (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 11/856,960

(22) Filed: Sep. 18, 2007

Related U.S. Application Data
(60) Provisional application No. 60/862,318, filed on Oct. 20, 2006.

(51) Int. Cl. A61F 2/06; B23H 3/00

(52) U.S. Cl. 623/1.15; 205/640; 205/674; 424/426; 623/1.42

(57) ABSTRACT

Medical devices, such as endoprostheses, and methods of making the devices are described. In some implementations, a stent has a surface region of magnesium with a protective surface layer of magnesium hydride obtained by hydrogen surface modification through an H-EIR process, offering enhanced corrosion resistance.

### CROSS-REFERENCE TO RELATED APPLICATIONS

This application claims priority under 35 USC § 119(e) to U.S. Provisional Patent Application Ser. No. 60/862,318, filed on Oct. 20, 2006, the entire contents of which are hereby incorporated by reference.

### TECHNICAL FIELD

The invention relates to medical devices, such as endoprostheses (e.g., stents).

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

### SUMMARY

The invention relates to medical devices, such as endoprostheses.

A new concept is described for using the relatively simple and cost-effective process of surface modification with hydrogen by electrochemical ion reduction (EIR) to tailor corrosion behavior of magnesium and magnesium alloy based stents. By application of the EIR process, there is formed on the stent surface a protective layer or coating of magnesium hydride (MgH₂), which is recognized to be a stable and electrically insulating compound.

According to one aspect of the disclosure, a medical stent device has a body comprising an erodible metal having a surface region of hydride formed by electrochemical reduction.

Preferred implementations of this aspect of the disclosure may include one or more of the following additional features. The erodible metal is magnesium, preferably comprising magnesium alloy, wherein the alloy includes one or more elements selected from the group consisting of: iron, calcium, zinc, iridium, platinum, ruthenium, tantalum, zirconium, silicon, boron, carbon, and alkali salts. The magnesium hydride region has a thickness of about 50 nm or more from the surface. The concentration of magnesium hydride decreases as a function of depth from the surface. The magnesium hydride region includes a therapeutic agent. The magnesium hydride region covers at least one of a luminal surface and an abluminal surface of the stent. The stent includes multiple hydride regions, at least two of which have contrasting thickness. The stent body is composed substantially of magnesium. The stent body includes magnesium on a nonerodible material.

According to another aspect of the disclosure, a method for forming a stent comprising providing a body comprising an erodible metal, and forming region of hydride by electrochemical reduction.

Preferred implementations of this aspect of the disclosure may include one or more of the following additional features. The erodible metal is magnesium. The method comprises the steps of: connecting the body as a cathode, immersing the body in an alkaline electrolyte solution, and exposing the stent to cathodic current pulses of the predetermined amplitude and duration. The method comprises incorporating a therapeutic agent into the hydride by providing the therapeutic agent in the electrolyte. The method comprises the step of immersing the body in an alkaline electrolyte solution of 0.01 M NaOH and 0.2 M Na2SO4. The method comprises masking the body to form the hydride region at a select locations on the body. The method comprises removing portions of the hydride region by laser ablation.

According to another aspect of the disclosure, a stent includes a body comprising an erodible metal including a continuous surface region of hydride.

Preferred implementations of this aspect of the disclosure may include one or more of the following additional features. The hydride region has a thickness of about 50 nm or more. The hydride includes a therapeutic agent. The hydride region is only on an abluminal surface of the stent. The body includes magnesium and a nonerodible metal. The thickness of the nonerodible metal is 75% or less of the thickness of the body.

According to still another aspect of the disclosure, a method of providing a therapeutic agent to a stent, comprises: providing a metal body for use in a stent, and processing the body by electrochemical reduction to form a hydride region on the body and incorporate therapeutic agent into the hydride region.

According to another aspect of the disclosure, a stent comprises a metal hydride including a therapeutic agent.

Implementation of the disclosure may result in one or more of the following advantages. A polymer-free coating, formed by electrochemical ion reduction (EIR), provides enhanced corrosion control for a biodegradable magnesium or magnesium alloy based stent. Also, as metal hydride complexes are known to be catalytically-active reducing agents, implementation of the disclosure may be expected that have a beneficial anti-oxidant effect in altering oxidation processes of LDL (low-density lipoprotein) cholesterol when the stent is placed in contact with blood flow.

The endoprostheses may not need to be removed from a lumen after implantation. The endoprostheses can have a low thrombogenecity and high initial strength. The endoprostheses can exhibit reduced spring back (recoil) after expansion. Lumens implanted with the endoprostheses can exhibit reduced restenosis. The rate of erosion of different portions of the endoprostheses can be controlled, allowing the endoprostheses to erode in a predetermined manner and reducing, e.g., the likelihood of uncontrolled fragmentation and embolization. For example, the predetermined manner of erosion can be from an inside of the endoprosthesis to an outside of the endoprosthesis, or from a first end of the endoprosthesis to a second end of the endoprosthesis. The controlled rate of erosion and the predetermined manner of erosion can extend the time the endoprosthesis takes to erode to a particular degree of erosion, can extend the time that the endoprosthesis can maintain patency of the passageway in which the endoprosthesis is implanted, can allow better control over the size of the released particles during erosion, and/or can allow the cells of the implantation passageway to better endothelialize around the endoprosthesis.

An erodible or bioerodible endoprosthesis, e.g., a stent, refers to an endoprosthesis, or a portion thereof, that exhibits substantial mass or density reduction or chemical transformation, after it is introduced into a patient, e.g., a human patient. Mass reduction can occur by, e.g., dissolution of the material that forms the endoprosthesis and/or fragmenting of the endoprosthesis. Chemical transformation can include oxidation/reduction, hydrolysis, substitution, and/or addition reactions, or other chemical reactions of the material from which the endoprosthesis, or a portion thereof, is made. The erosion can be the result of a chemical and/or biological interaction of the endoprosthesis with the body environment, e.g., the body itself or body fluids, into which the endoprosthesis is implanted and/or erosion can be triggered by applying a triggering influence, such as a chemical reactant or energy to the endoprosthesis, e.g., to increase a reaction rate. For example, an endoprosthesis, or a portion thereof, can be formed from an active metal, e.g., Mg or Ca or an alloy thereof, and which can erode by reaction with water, producing the corresponding metal oxide and hydrogen gas (a redox reaction). For example, an endoprosthesis, or a portion thereof, can be formed from an erodible or bioerodible polymer, an alloy, and/or a blend of erodible or bioerodible polymers which can erode by hydrolysis with water. The erosion occurs to a desirable extent in a time frame that can provide a therapeutic benefit. For example, in embodiments, the endoprosthesis exhibits substantial mass reduction after a period of time when a function of the endoprosthesis, such as support of the lumen wall or drug delivery, is no longer needed or desirable. In particular embodiments, the endoprosthesis exhibits a mass reduction of about 10 percent or more, e.g. about 50 percent or more, after a period of implantation of one day or more, e.g. about 60 days or more, about 180 days or more, about 600 days or more, or 1000 days or less. In embodiments, only portions of the endoprosthesis exhibit erodibility. For example, an exterior layer or coating may be non-erodible, while an interior layer or body is erodible. In some embodiments, the endoprosthesis includes a substantially non-erodible coating or layer of a radiopaque material, which can provide long-term identification of an endoprosthesis location.

Erosion rates can be measured with a test endoprosthesis suspended in a stream of Ringer's solution flowing at a rate of 0.2 m/second. During testing, all surfaces of the test endoprosthesis can be exposed to the stream. For the purposes of this disclosure, Ringer's solution is a solution of recently boiled distilled water containing 8.6 gram sodium chloride, 0.3 gram potassium chloride, and 0.33 gram calcium chloride per liter of solution.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification will control. In addition, the materials, methods, and examples are illustrative only and not intended to be limiting. Other features and advantages will be apparent from the following detailed description, and/or from the claims.

### DESCRIPTION OF DRAWINGS

FIG. 1 is a perspective view of an implementation of an expanded stent.

FIGS. 2-2B are cross sectional views of a stent in a body lumen schematically illustrating erosion.

FIG. 3 is a schematic cross section through the body of a stent illustrating composition as a function the thickness of the body.

FIGS. 5 and 5A are cross-section views of an embodiment of a stent before and after erosion, respectively.

FIGS. 6 and 6A are cross sectional views of an embodiment of a stent before and after erosion, respectively.

Like reference symbols in the drawing indicate like elements.

### DETAILED DESCRIPTION

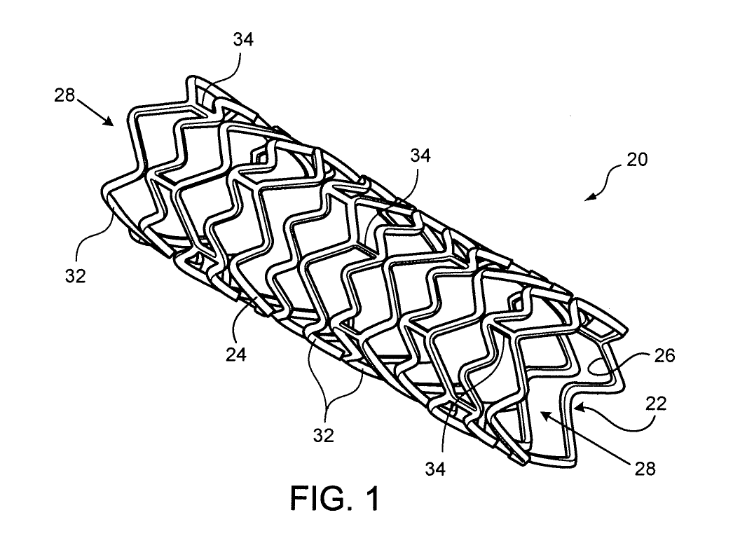

Referring to FIG. 1, a stent 20 has the form of a tubular body 22 defining an outer (abluminal) wall surface 24 and an inner (luminal) wall surface 26. The inner wall surface defines a central lumen 28. The stent tubular body 22 is defined by a plurality of bands 32 and a plurality of connectors 34 extending between and connecting adjacent bands. During use, bands 32 are caused to expand from an initial, small outer diameter to a relatively larger outer diameter, moving the outer wall surface 24 of stent 20 into contact with a surrounding wall of a vessel, thereby to assist in maintaining the patency of the vessel. Connectors 34 provide stent 20 with flexibility and conformability that allow the stent to adapt to the contours of the vessel.

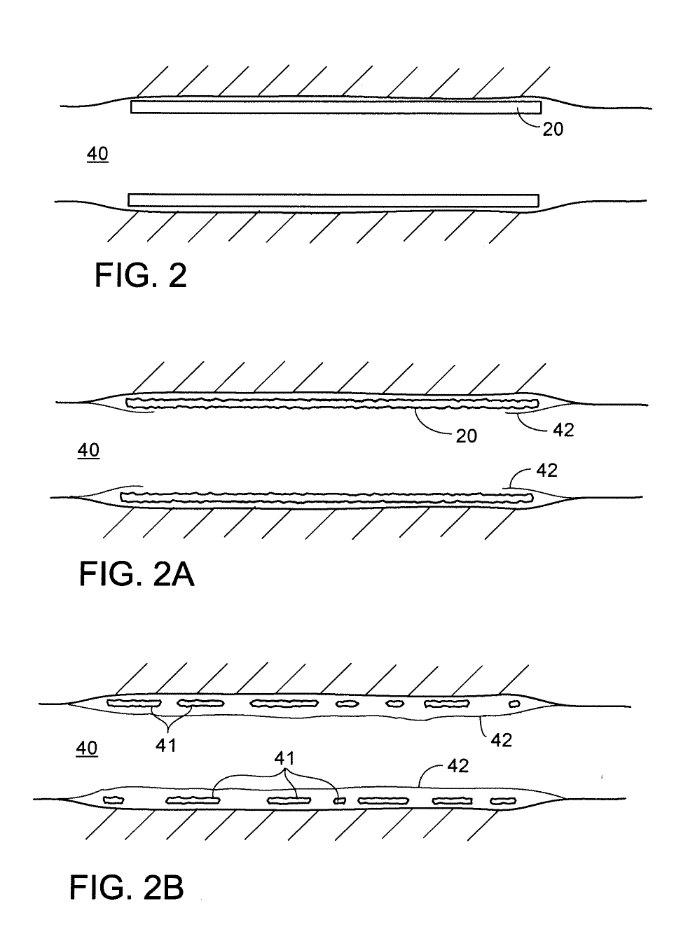

Referring as well to FIGS. 2-2B, the stent 20 is formed such that it erodes over time after being implanted in a body lumen. Referring particularly to FIG. 2, the stent 20 is placed in a body lumen 40, such as a vascular lumen, e.g. a coronary artery. Typically, the stent is delivered into the lumen on a catheter in a collapsed state and expanded into contact with the lumen wall by inflation of a balloon. Alternatively, the stent is formed of a metal that self-expands by release of its internal elastic forces. Stent delivery is further discussed in Heath, U.S. Pat. No. 5,725,570. Initially, the stent has a metallic body of characteristic thickness. Referring particularly to FIGS. 2A and 2B, over time the thickness of the stent is reduced as the stent erodes. The continuous nature of the stent body is interrupted as it is eroded into fragments 41. The stent, as a body, and/or as fragments, is endothelialized 42 by the lumen wall.

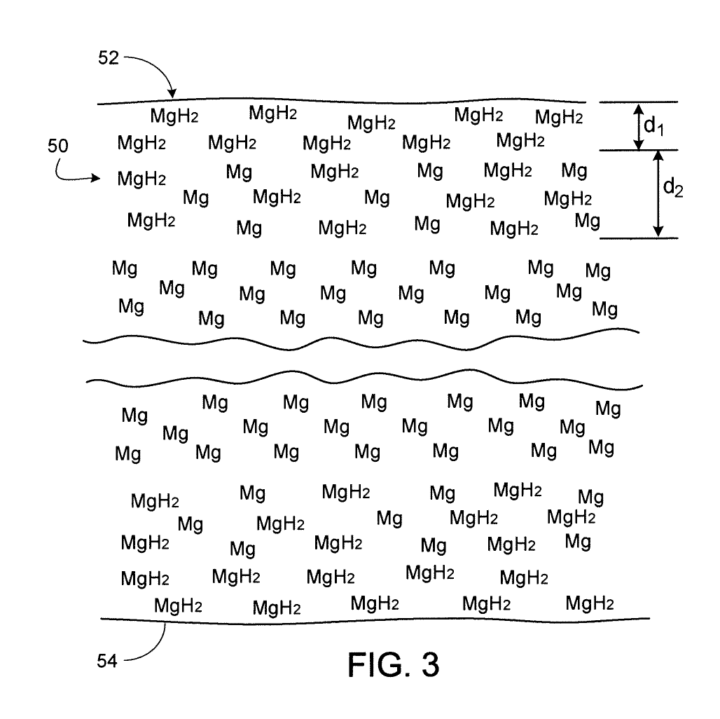

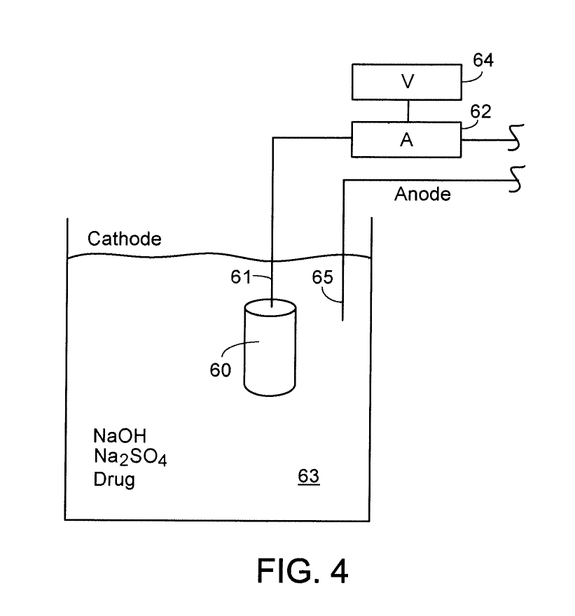

Referring to FIG. 3, the stent is formed of an erodible metal such as magnesium, e.g., pure magnesium or a magnesium alloy, that has been treated to tailor the timing and pattern of erosion. In the example illustrated in FIG. 3, the stent body 50 is formed of magnesium that has been modified proximate its luminal surface 52 and its abluminal surface 54 to include magnesium hydride. In particular, the stent body is substantially magnesium hydride from the surfaces to a depth d₁. From a depth d₁ to d₂, the concentration of magnesium hydride decreases. Below the depth d₂, the stent body is substantially magnesium. The hydride erodes at a substantially reduced rate compared to the underlying magnesium and forms a barrier through which body fluid must pass, e.g. by diffusion, that reduces the exposure of the magnesium to body fluid and thus the rate at which the magnesium erodes. The rate of erosion can be controlled by selecting the thicknesses d₁, d₂ of the hydride-containing regions and/or the area of the stent body covered by the magnesium hydride regions. The magnesium hydride regions are formed continuously with the stent body, typically penetrating into the bulk of the magnesium body and thus are tightly bound, which enhances stability of the hydride and reduces the likelihood of premature delamination.

Referring to FIG. 4, the hydride is formed by an electrochemical process in which hydrogen ions are reduced from an alkaline solution. A body 60 of magnesium for use in a stent is connected as a cathode 61 to a power source 62 and immersed in an alkaline electrolyte 63 of, e.g., 0.01 M NaOH (sodium hydroxide) and Na2SO4 (disodium sulfate), in which an anode 65 is also immersed. The power source 62 includes a controller 64 to control the cathodic current amplitude, pulse width, and overall duration, to control the nature and depth of the hydride regions. The electrochemical process is a rapid, one step technique for formation of the hydride. The formation of an oxide, which is relatively less effective in controlling erosion than the hydride, can be discouraged by purging the electrolyte with nitrogen. Suitable processes, such as electrochemical ion reduction (EIR), and characterizations of hydrides are described in Bakkar et al., Corrosion Science, 47:1211-1225 (2005), Fischer et al., Journal of Less Common Metals 172-174:808-815 (1991), and U.S. Pat. No. 6,291,076. In embodiments, the hydrogen content as a function of depth from the surface can be determined by SIMS. In particular embodiments, substantially increased hydrogen content is observed in the first 50 nm or more from the surface, e.g. the first 50-800 nm, e.g. the first 200 nm or less, with lower moderately decreasing hydrogen counts observed at greater depths. In embodiments, the presence of hydrogen is not substantially detected at depths greater than about 10 microns, e.g. not greater than about 5 microns or 2 microns.

The hydride material can as well be a depository of therapeutic substances which diffuse through the hydride matrix to treat the body lumen. Continuing to refer to FIG. 4, the therapeutic agent or “drug” can be incorporated into the hydride during formation. In particular, the therapeutic agent can be dissolved in the electrolyte, e.g. as a salt to provide an ionic form, and the controller used to modify the pulses to the body such that the therapeutic agent is drawn to the stent. For example, polarity of the pulse can be modified to alternately draw therapeutic agent to the stent body and form the hydride such that a controlled amount of therapeutic agent is incorporated as a function of depth.

Suitable biodegradable metals include metals effective for stent use, such as iron and particularly magnesium, including magnesium alloys and composites, which may be formulated, e.g., with biocompatible elements such as iron, calcium, zinc, iridium, platinum, ruthenium, tantalum, zirconium, silicon, boron, carbon, alkali salts, and other suitable materials. Alloys include AZ91—Mg (Mg; 9% Al; 1% Zn; 0.2% Mn). Other alloys are described in Metals Handbook, 9th Edition, Vol. 13, Corrosion, 1987 (e.g., Table 4 of typical magnesium alloy compositions). Erodible metal materials are further described in Bolz U.S. Pat. No. 6,287,332 (e.g. sodium-magnesium alloys), Heublien U.S. Patent Application No. 2002/000406, and Park, Science and Technology of Advanced Materials, 2:73-78 (2001) (e.g. Mg—X—Ca alloys such as Mg—Al—Si—Ca, and Mg—Zn—Ca alloys).

The hydride can be provided on both luminal (inner) and abluminal (outer) surfaces, as illustrated in FIG. 3, or on just the luminal or just the abluminal surface. The hydride can also be provided in intermittent select locations on one or more of the surfaces. The surfaces can be masked (e.g. with polymer) during the electrochemical process, e.g. with a removable polymer mandrel (e.g. polycarbonate), or the hydride can be selectively removed after formation, e.g. by laser ablation.

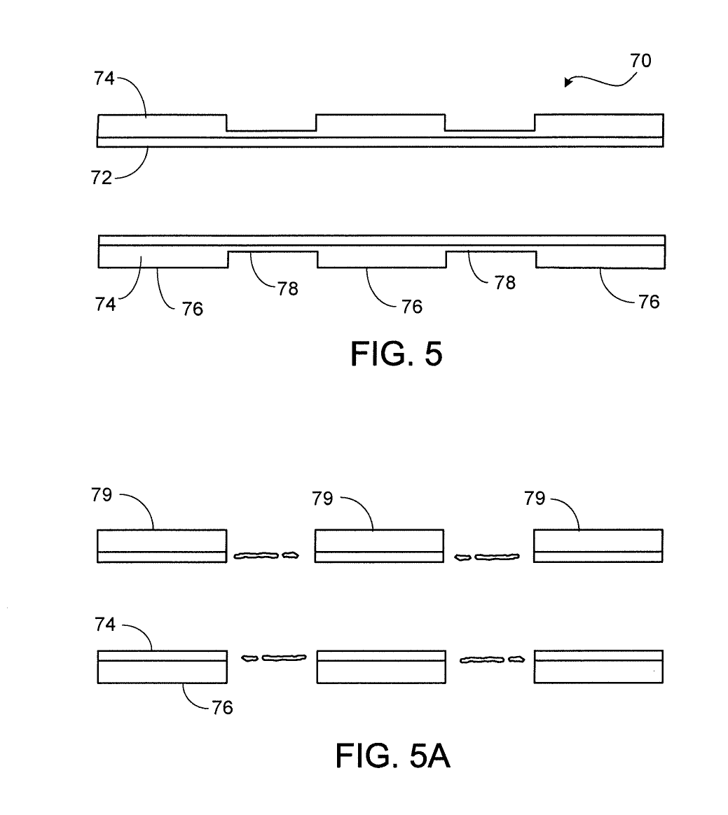

Referring to FIGS. 5 and 5A, the thickness of the hydride regions can be varied along the stent. Referring particularly to FIG. 5, a stent 70 has an erodible body 72 with a hydride 74 on its abluminal surface. The body 72 has intermittent hydride regions of greater thickness 76 and regions of reduced thickness 78. Referring particularly to FIG. 5A, after erosion in the lumen, the body 72 erodes at a greater rate at locations corresponding the regions of reduced hydride thickness 78, resulting in a series of shorter rings 79, which reduce interference with the lumen's natural flexibility as the stent erodes.

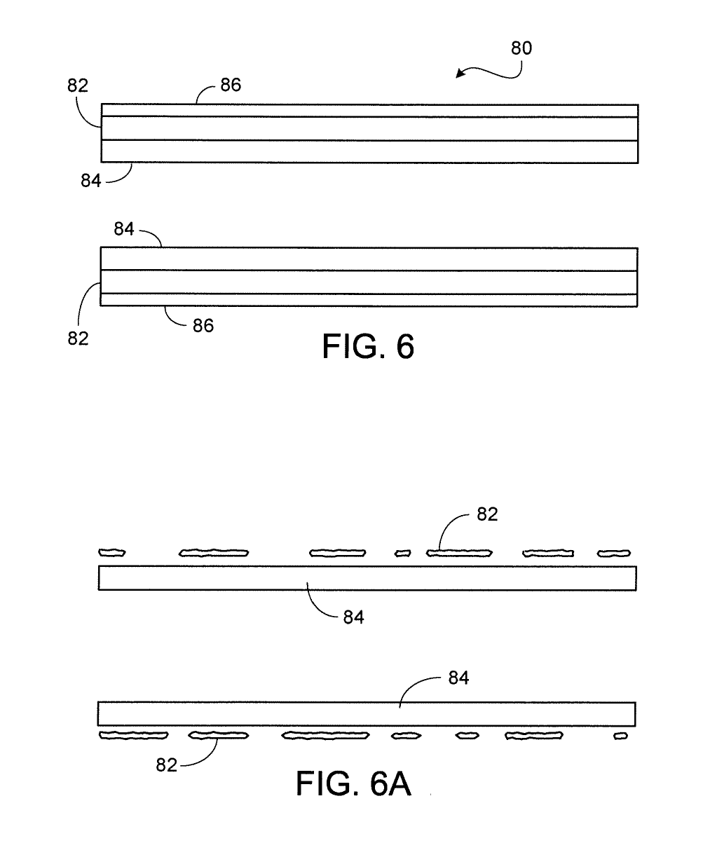

Referring to FIGS. 6 and 6A, in embodiments, the stent is a composite stent including an erodible material and a nonerodible material. Referring particularly to FIG. 6, a stent 80 includes an erodible layer 82, e.g. a magnesium layer, over a nonerodible layer 84, e.g. stainless steel. The erodible layer 82 includes a hydride 86 to control the erosion and/or drug delivery. Referring to FIG. 6A, after erosion, the nonerodible material 84 remains, but the erodible layer 82 is eroded and the hydride 86 substantially degrades. The nonerodible material that remains is much thinner than a completely nonerodible stent, resulting in a more flexible structure remaining in the body. As a result, the composite structure can have increased strength by use of conventional nonerodible stent materials but results in a much thinner nonerodible body remaining in the lumen after the erodible material has been eroded. Also, by causing the stent to erode preferentially from the inner surface, as compared to the outer surface, the diameter of the center lumen or passageway increases over time, which can facilitate passage, e.g., of medical instruments and devices during subsequent procedures. In embodiments, the nonerodible layer is about 75% or less of the initial stent thickness, e.g. about 50% or less or about 35% or more. In embodiments, the hydride can be used as a metal drug eluting coating, e.g. over a conventional non-eroding metal stent. The hydride can be a hydride of a nonerodible or erodible metal and formed by electrochemical reduction. The coating can be, e.g., about 10 microns thick or less.

In embodiments, the stent has mechanical properties that allow a stent including a composite material to be compacted, and then subsequently expanded to support a vessel. In some implementations, stent 20 can have an ultimate tensile yield strength (YS) of about 20-150 ksi, greater than about 15% elongation to failure, and a modulus of elasticity of about 10-60 msi. When stent 20 is expanded, the material can be stretched to strains on the order of about 0.3. Examples of materials suitable for use in the tubular body of a stent include stainless steel (e.g., 316L, BioDur® 108 (UNS S29108), and 304L stainless steel, and an alloy including stainless steel and 5-60% by weight of one or more radiopaque elements (e.g., platinum, iridium, gold, tungsten, etc.) (PERSS®) as described in U.S. Patent Publication Nos. 2003-0018380-A1, 2002-0144757-A1, and 2003-0077200-A1), Nitinol (a nickel-titanium alloy), cobalt alloys such as Elgiloy, L605 alloys, MP35N, titanium, titanium alloys (e.g., Ti-6Al-4V, Ti-50Ta, Ti-10Ir), platinum, platinum alloys, niobium, niobium alloys (e.g., Nb-1Zr), Co-28Cr-6Mo, tantalum, and tantalum alloys. Other examples of materials are described in commonly assigned U.S. application Ser. No. 10/672,891, filed Sep. 26, 2993, and entitled “Medical Devices and Methods of Making Same;” and U.S. application Ser. No. 11/035,316, filed Jan. 3, 2005, and entitled “Medical Devices and Methods of Making Same.” Other materials include elastic biocompatible metals such as a superelastic or pseudo-elastic metal alloy, as described, e.g., in Schetsky, L. McDonald, “Shape Memory Alloys,” Encyclopedia of Chemical Technology (3rd ed.), John Wiley & Sons, 1982, vol. 20. pp. 726-736; and commonly assigned U.S. application Ser. No. 10/346,487, filed Jan. 17, 2003. In some embodiments, the stent body may include one or more materials that enhance visibility by MRI (magnetic resonance imaging). Examples of MRI-enhancing materials include non-ferrous metals (e.g., copper, silver, platinum, or gold) and non-ferrous metal-alloys containing paramagnetic elements (e.g., dysprosium or gadolinium) such as terbium-dysprosium. Alternatively or additionally, stent body 22 can include one or more materials having low magnetic susceptibility to reduce magnetic susceptibility artifacts, which during imaging can interfere with imaging of tissue, e.g., adjacent to and/or surrounding the stent. Low magnetic susceptibility materials include those described above, such as tantalum, platinum, titanium, niobium, copper, and alloys containing these elements.

According to one implementation, a generally imperforate tubular body member of a magnesium or magnesium alloy based stent is preferentially treated upon its outer surface by surface deformation with hydrogen by electrochemical ion reduction (EIR) to convert magnesium at the outer (abluminal) wall surface to a protective layer of magnesium hydride. Bands and connectors of the stent are then formed by cutting the tubular body member. For example, selected portions of the tube can be removed to form the bands 32 and connectors 34, e.g. by laser ablation, or by laser cutting as described in U.S. Pat. No. 5,780,807. In certain implementations, a liquid carrier, such as a solvent or an oil, is flowed through the lumen of the tube during laser cutting. The carrier can prevent dross formed on one portion of the tube from re-depositing on another portion and/or can reduce formation of recast material on the tube. Other methods for removing portions of the tube can also be used, such as mechanical machining (e.g., micro-machining), electrical discharge machining (EDM), and photoetching (e.g., acid photoetching). In some implementations, after bands and connectors are formed, areas of the tube affected by the cutting operation above can be removed. For example, laser machining of bands 32 and connectors 34 can leave a surface layer of melted and resolidified material and/or oxidized metal that can adversely affect mechanical properties and performance of stent 20. The affected areas can be removed mechanically (such as by grit blasting or honing) and/or chemically (such as by etching or electropolishing). However, by use of laser ablation, in particular with ultrashort lasers, melting and the resultant debris can be virtually eliminated, making further polishing unnecessary. Thus in some implementations, the tubular member can be near net shape configuration these steps are performed. “Near-net size” means that the tube has a relatively thin envelope of material required to be removed to provide a finished stent. In some implementations, the tube is formed less than about 25% oversized, e.g., less than about 15%, 10%, or 5% oversized. In other implementations, the unfinished stent can next be finished to form stent 20, for example, by electropolishing to a smooth finish. Since the unfinished stent can be formed to near-net size, relatively little of the unfinished stent need to be removed to finish the stent. As a result, further processing, which can damage the stent, and consumption of costly materials can be reduced. In some implementations, about 0.0001 inch of the stent material can be removed by chemical milling and/or electropolishing to yield a stent.

As described above, therapeutic agents can be incorporated in the hydride. Therapeutic agents can also be provided on the surface of the hydride. Suitable therapeutic agents are described in U.S. Pat. No. 5,674,242 and U.S. application Ser. No. 09/895,415, filed Jul. 2, 2001; and Ser. No. 10/232,265, filed Aug. 30, 2002. The therapeutic agents, drugs, or pharmaceutically active compounds can include, for example, anti-thrombogenic agents, antioxidants, anti-inflammatory agents, anesthetic agents, anti-coagulants, and antibiotics.

The stent can be of a desired shape and size (e.g., coronary stents, aortic stents, peripheral vascular stents, gastrointestinal stents, urology stents, and neurology stents). Depending on the application, the stent can have a diameter of between, for example, 1 mm to 46 mm. In certain embodiments, a coronary stent can have an expanded diameter of from about 2 mm to about 6 mm. In some embodiments, a peripheral stent can have an expanded diameter of from about 5 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. Stent 20 can be balloon-expandable, self-expandable, or a combination of both (e.g., U.S. Pat. No. 5,366,504). In use, the stent can be used, e.g., delivered and expanded, using a catheter delivery system. Catheter systems are described in, for example, U.S. Pat. Nos. 5,195,969; 5,270,086; and 6,726,712. Stents and stent delivery are also exemplified by the Radius® or Symbiot® systems, available from Boston Scientific Scimed, Maple Grove, Minn. In some embodiments, stent can be formed by fabricating a wire including the composite material, and knitting and/or weaving the wire into a tubular member. The Stent can be a part of a covered stent or a stent-graft. In other implementations, stent 20 can include and/or be attached to a biocompatible, non-porous or semi-porous polymer matrix made of polytetrafluoroethylene (PTFE), expanded PTFE, polyethylene, urethane, or polypropylene.

Other embodiments are within the claims.

What is claimed is:

1. A medical stent device having a body comprising an erodible metal having a surface region of hydride formed by electrochemical reduction.

2. The medical device of claim 1, wherein the erodible metal is magnesium.

3. The medical device of claim 2, wherein the magnesium comprises magnesium alloy.

4. The medical device of claim 2, wherein the alloy includes one or more elements selected from the group consisting of: iron, calcium, zinc, iridium, platinum, ruthenium, tantalum, zirconium, silicon, boron, carbon, and alkali salts.

5. The medical device of claim 2 wherein the magnesium hydride region has a thickness of about 50 nm or more from the surface.

6. The medical device of claim 2 wherein the concentration of magnesium hydride decreases as a function of depth from the surface.

7. The medical device of claim 2 wherein the magnesium hydride region includes a therapeutic agent.

8. The medical device of claim 2, wherein the magnesium hydride region covers at least one of a luminal surface and an abluminal surface of the stent.

9. The medical device of claim 2, wherein the stent includes multiple hydride regions, at least two of which have contrasting thickness.

10. The medical device of claim 2 wherein the stent body is composed substantially of magnesium.

11. The medical device of claim 2 wherein the stent body includes magnesium on a nonerodible material.

12. A method for forming a stent comprising providing a body comprising an erodible metal, and forming region of hydride by electrochemical reduction.

13. The method of claim 12 wherein the erodible metal is magnesium.

14. The method of claim 12, comprising the steps of:
connecting the body as a cathode,
immersing the body in an alkaline electrolyte solution, and
exposing the stent to cathodic current pulses of the predetermined amplitude and duration.

15. The method of claim 14 comprising incorporating a therapeutic agent into the hydride by providing the therapeutic agent in the electrolyte.

16. The method of claim 15, comprising the step of:
immersing the body in an alkaline electrolyte solution of 0.01 M NaOH and 0.2 M Na₂SO₄.

17. The method of claim 14 comprising masking the body to form said hydride region at a select locations on the body.

18. The method of claim 14 comprising removing portions of said hydride region by laser ablation.

19. A stent including a body comprising an erodible metal including a continuous surface region of hydride.

20. The medical device of claim 19 wherein the hydride region has a thickness of about 50 nm or more.

21. The medical device of claim 19 wherein the hydride includes a therapeutic agent.

22. The stent of claim 19 including the hydride region is only on an abluminal surface of the stent.

23. The stent of claim 19 wherein the body includes said magnesium and a nonerodible metal.

24. The stent of claim 23 in which the thickness of the nonerodible metal is 75% or less of the thickness of the body.

25. A method of providing a therapeutic agent to a stent, comprising:
providing a metal body for use in a stent, and
processing the body by electrochemical reduction to form a hydride region on the body and incorporate therapeutic agent into said hydride region.

26. A stent, comprising a metal hydride including a therapeutic agent.
